(19) United States
(12) Patent Application Publication
(10) Pub. No.: US 2007/0136958 A1
(43) Pub. Date: Jun. 21, 2007

(54) Hair dye kit

(76) Inventor: Amy E. Draghiceanu, Canton, GA (US)

Correspondence Address: GEORGE R. REARDON, 3356 STATION COURT, LAWRENCVILLE, GA, 30044, US

(21) Appl. No.: 11/304,866

(22) Filed: Dec. 15, 2005

(51) Int. Cl. A61K 8/00

(52) U.S. Cl. 8/405

(57) ABSTRACT

The general purpose of the present invention is to provide a convenient to use hair dye kit. The present invention allows users to dye hair by using a hair dye kit that eliminates the need for messy mixing of the elements needed to dye hair. The present invention is comprised of a multiple chamber syringe containing the ingredients needed to be mixed in order to dye hair. Each chamber is further comprised of a removable cap attached to an outlet tip that is operable for release of the chamber contents when the common plunger is engaged to create a controled release of the contents of each chamber. The present invention is further comprised of a removable mixing tip that is operable for engaging the outlet tips of each chamber, once the caps are removed, such that the contents from each chamber flows into the mixing tip, are properly combined, and flow out of the orifice at the dispensing end of the mixing tip and onto the targeted portion of the hair.

### FIELD OF THE INVENTION

The invention is generally directed to hair coloring tools. More particularly the invention relates to kits for the dying of hair.

### BACKGROUND OF THE INVENTION

Historically, hair coloring has been done by women for centuries in order to cover graying hair, or to change the appearance of their hair. Men, in increasing numbers, have colored their hair for similar reasons. Originally, hair coloring dyes were based on naturally occurring dyes such as those produced by plants or various minerals. Today, there are a multitude of hair coloring products, including coloring shampoos, rinses, semi-permanent hair coloring compositions, and permanent hair coloring compositions.

Hair dying is often a difficult, messy and complex procedure even for hair care professionals. Frequently, today's hair coloring products require the mixing together of two or more components prior to application to the user's hair, e.g., a liquid or paste hair coloring composition and a liquid developer or fixing solution. Hair care professionals are usually highly skilled in applying hair dying compositions in order to be able to consistently and repeatedly blend a diverse number of coloring, coupling and developing agents to provide a client with a desired hair color. A hair care professional typically mixes the required quantities of hair coloring composition and fixing solution in a bowl or similar container. The mixed coloring composition is then applied to the user's hair using a brush designed for that purpose.

Coloring may be done on all of the user's hair or only to portions of the hair such as in “streaking” the hair. A week or so after all of the user's hair has been colored, touch-up coloring is required to color the hair roots that have grown out. This is usually a time consuming process, whether done by the user at home, or by a hair care professional.

Attempts to address these and other problems include U.S. Published Patent Application No. 2005/0067320, inventor Deborah T. Rodriguez, published on Mar. 31, 2005, discloses a hair roots coloring kit which includes a plurality of kit components carried in a drawstring bag for mixing and applying standard hair coloring materials to the uncolored outgrown roots of a user's hair. One or more measuring devices such as a measuring cup, a syringe, or an eyedropper are used for measuring the hair coloring materials to produce a matching color to previously colored hair shafts of the hair. A reusable mixing bowl or a plurality of disposable mixing bowls holds the measured hair coloring materials. A reusable stirring stick or a plurality of disposable stirring sticks are provided for mixing together the measured hair coloring materials in the mixing bowl. An applicator brush is used for applying the mixed matching color of hair coloring composition to the uncolored hair roots. A pair of protective gloves is provided to protect the user's hands during application of the hair coloring composition to the hair roots. A cleaning brush is used to aid in cleaning the kit components following use.

U.S. Pat. No. 6,440,175, issued to Virgil E. Stanley, III on Aug. 27, 2002 discloses a hair coloring kit that enables a person to mix or formulate a plurality of different hair color compositions. The kit includes a base composition or hair spray, a group of color concentrates, and a dispenser. One or more of the color concentrates is mixed with the base composition in the dispenser to form a hair coloring composition of a particular color. Thereafter, the formed hair coloring composition can be sprayed onto the hair of a person.

U.S. Pat. No. D465,612, issued to Phyllis L. Boyd on Nov. 12, 2002, illustrates the ornamental design for a hair dye kit.

U.S. Pat. No. 6,357,450, issued to Andrew Paice on Mar. 19, 2002, discloses a hair dye applicator for mixing dye gel and peroxide as needed. The hair dye applicator includes a housing having a top wall, a bottom wall, a front wall, a back wall and a pair of lateral side walls. A cartridge comprises a first tubular member and a second tubular member coupled together. Each of the tubular members has an open first end and a closed second end. Each of the second ends of the tubular member has a hole extending therein. An annular lip is integrally coupled to the second end and encompasses the holes in the second end. Dye gel is positioned in one of the tubular members and peroxide activator is positioned in the other of the tubular members. A coupling member removably couples the first end of the cartridge to the front wall of the housing. A generally hollow rod and has open first and second ends. A bracket member releasably secures the first end of the rod to the second end of the cartridge. The second end of the rod extends into and is fluidly coupled to a manifold. A plurality of conduits is fluidly coupled to and extends away from the front side of the manifold. An actuator assembly forces the gel dye and peroxide activator out of the cartridge and into the manifold.

U.S. Pat. No. 5,554,197, issued to Anthony Assini on Sep. 10, 1996, discloses methods and systems for hair dying. The system includes at least two reservoirs containing different hair dying components in predetermined concentrations, the containers including outlets having cross-sectional areas which are sized relative to the concentration of the component contained in the reservoir and the amount of that component needed to react with the remaining components. The measuring device calibrated with respect to the concentrations of the hair dye components and the cross-sectional areas of their respective reservoir outlets, permits rapid and accurate dispensing of the hair dye components in the proper quantities. In a preferred arrangement, the measuring device is provided on a blending board so that the components can be blended together immediately upon dispensing.

While these patents and published patent application and other previous methods have attempted to solve the above mentioned problems, none have utilized or disclosed a hair dye kit that contains a multiple chamber hair dye applicator with a removable mixing tip and caps for the dispensing end of the multiple chamber hair dye applicator, with the multiple chambers containing the necessary ingredients to dye hair.

Therefore, a need exists for a convenient, preloaded hair dye kit that is operable for full or partial use in dying hair without the need for messy preparation. The hair dye kit according to the present invention substantially departs from the conventional concepts and designs of the prior art. It can be appreciated that there exists a continuing need for a hair dye kit which can be used commercially. In this regard, the present invention substantially fulfills these objectives.

The foregoing patent and other information reflect the state of the art of which the inventor is aware and are tendered with a view toward discharging the inventor's acknowledged duty of candor in disclosing information that may be pertinent to the patentability of the present invention. It is respectfully stipulated, however, that the foregoing patent and other information do not teach or render obvious, singly or when considered in combination, the inventor's claimed invention.

### BRIEF SUMMARY OF THE INVENTION

The general purpose of the present invention, which will be described subsequently in greater detail, is to provide a convenient to use hair dye kit. In particular, the present invention relates to a hair dye kit that eliminates the need for messy mixing of the elements needed to dye hair.

The present invention is comprised of a multiple chamber syringe containing the ingredients needed to be mixed in order to dye hair, e.g. developer in one chamber and colorant in the other chamber. Each chamber is further comprised of an outlet tip that is operable for release of its contents when the common plunger is engaged to create a controled release of the contents of each chamber. In some embodiments the number of chambers may be more that two, e.g. three chambers, four chambers. The contents of these additional chambers may be the same ingredients, or may be different ingredients, e.g. conditioner, additional hair dye.

The present invention may be further comprised of a cap for each chamber outlet tip. Each cap is operable for closing the outlet tip such that none of the contents can be released. Each cap is operable for being completely removed from each chamber outlet tip.

The present invention is further comprised of a mixing tip that is preferably removable, but may be permanently affixed in an embodiment having no caps. The removable mixing tip is operable for engaging the outlet tips of each chamber, once the caps are removed, such that the contents from each chamber flows into the mixing tip, are properly combined, and flow out of the orifice at the dispensing end of the mixing tip and onto the targeted portion of the hair.

In some embodiments of the present invention, further elements may be included in the kit, e.g. gloves, combs, brushes, additional caps, additional mixing tips, additional dual chamber syringes, hair dye remover, a mirror, directions. Additionally, in some embodiments of the present invention, traditional hair coloring elements may also be included, such as those currently on the market by L'Oreal, Clairol, Combe, Revlon, Garnier and others. Typically these traditional hair coloring elements may include developer, colorant, conditioner, sealer and the like. The present invention may be used for touching up the roots of hair, streaking hair, or coloring a full head of hair, depending on the embodiment of the invention.

An advantage of the present invention is that it is easy to use.

Another advantage of the present invention is that it is convenient to use.

Another advantage of the present invention is that it can be manufactured from readily available materials.

Another advantage of the present invention is that it is a cost-effective alternative to existing methods.

Another advantage of the present invention is that it allows for partial use and storage for further use at a later date.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further objects and advantages of the present invention will be apparent from the following detailed description of a presently preferred embodiment which is illustrated schematically in the accompanying drawings.

### DESCRIPTION OF THE SEVERAL VIEWS OF THE DRAWINGS

Other advantages and features of the invention are described with reference to exemplary embodiments, which are intended to explain and not to limit the invention, and are illustrated in the drawings in which:

FIG. 1 is a perspective view of a hair dye kit without caps, according to an embodiment of the present invention.

FIG. 2 is a perspective view of a hair dye kit without caps with a right angle mixing tip, according to an embodiment of the present invention.

FIG. 3 is a perspective view of a hair dye kit with caps, gloves, brush and a mixing tip, according to an embodiment of the present invention.

FIG. 4 is a perspective view of a hair dye kit with caps, gloves, brush and a right angle mixing tip, according to an embodiment of the present invention.

FIG. 5 is a perspective view of a hair dye kit with caps, gloves, brush and a mixing tip, illustrating the caps removed, according to an embodiment of the present invention.

FIG. 6 is a perspective view of a hair dye kit with caps, gloves, brush and a right angle mixing tip, illustrating the caps removed, according to an embodiment of the present invention.

### DETAILED DESCRIPTION OF THE INVENTION

Before explaining the disclosed embodiment of the present invention in detail it is to be understood that the invention is not limited in its application to the details of the particular arrangement shown since the invention is capable of other embodiments. Also, the terminology used herein is for the purpose of description and not of limitation.

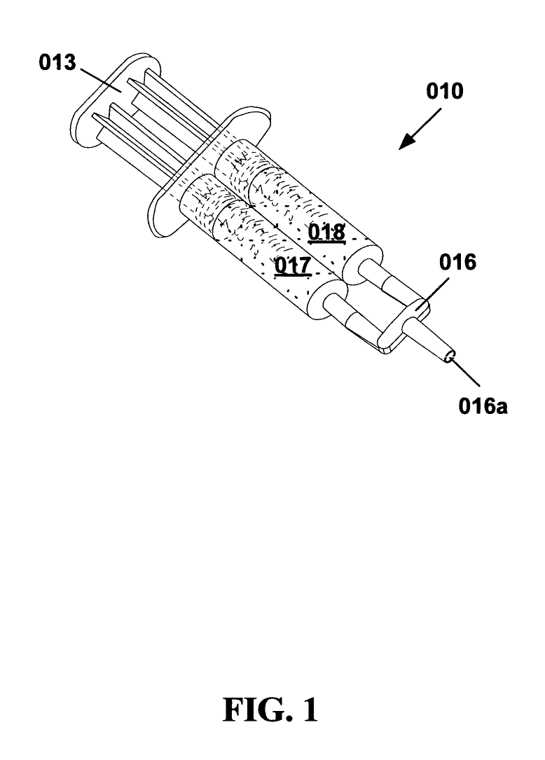

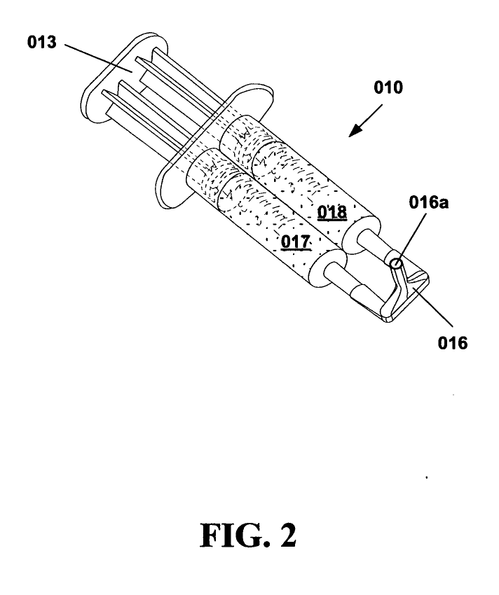

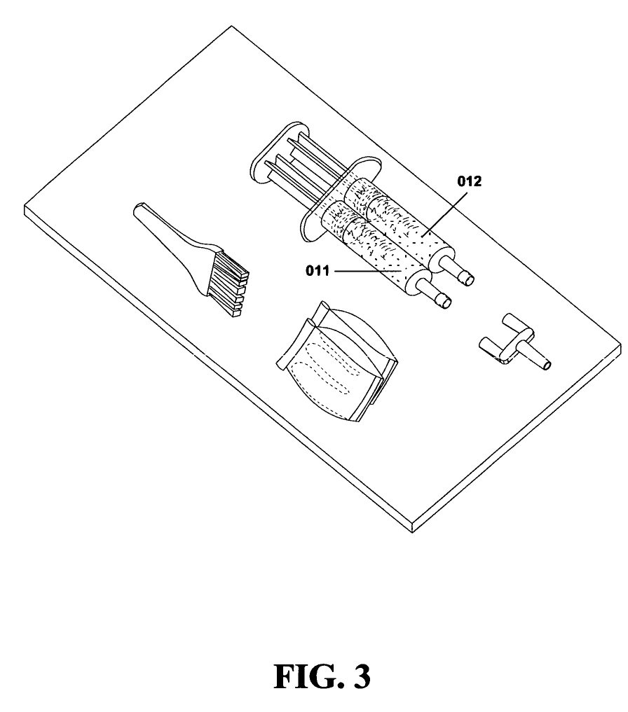

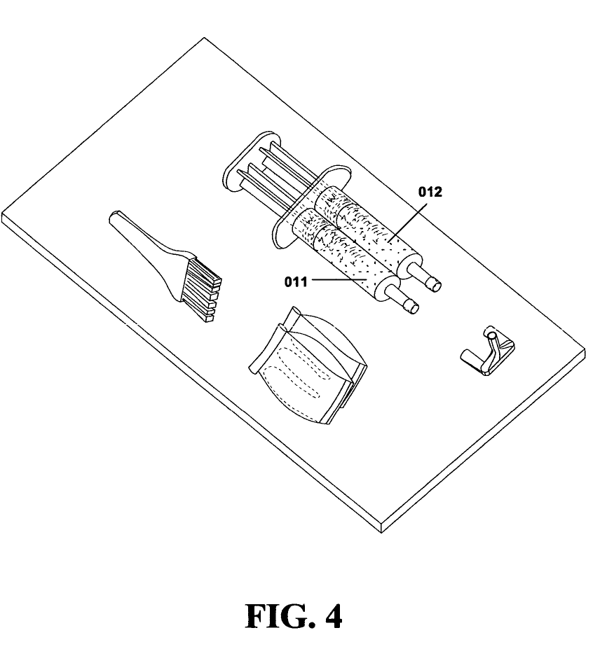

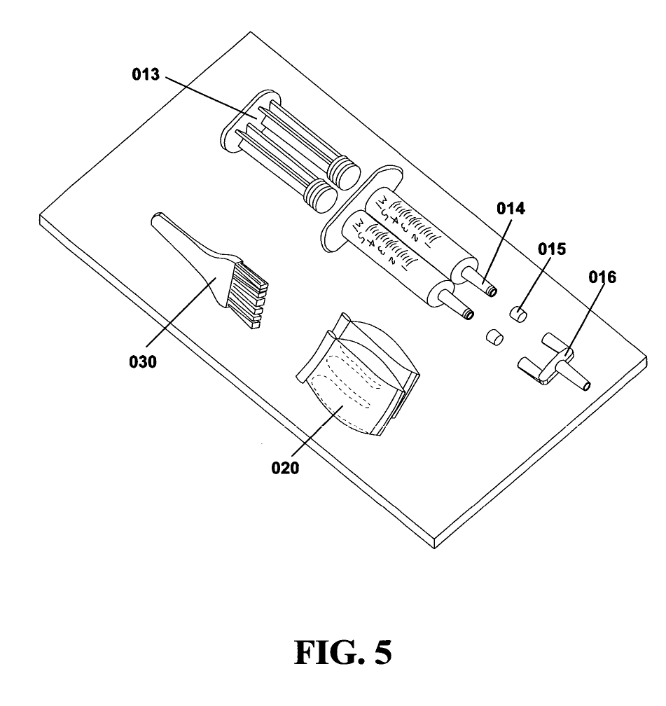

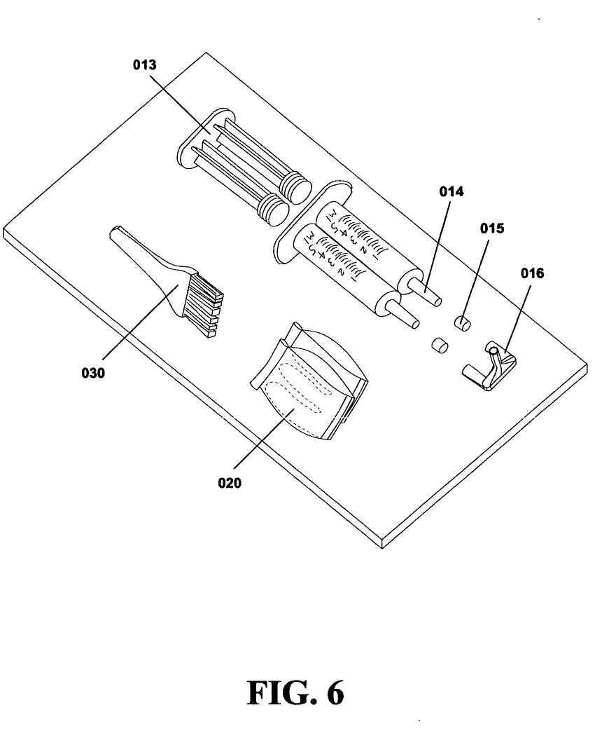

Referring now to FIG. 1, FIG. 2, FIG. 3, FIG. 4, FIG. 5, and FIG. 6, multiple embodiments of a hair dye kit are illustrated. FIG. 1 and FIG. 2 illustrate embodiments comprised of multiple chamber syringe 010. In these embodiments, multiple chamber syringe 010 is comprised of first chamber 011, second chamber 012, plunger 013, tips 014, and mixing tip 016. Contained within first chamber 011 is first composition 017. Contained within second chamber 012 is second composition 018. Mixing tip 016 is further comprised of dispensing orifice 016a. In FIG. 2, the nozzle of mixing tip 016 is configured to be at an angle, preferably a right angle.

FIG. 3 and FIG. 4 illustrate embodiments comprised of multiple chamber syringe 010, gloves 020 and brush 030. In these embodiments, multiple chamber syringe 010 is further comprised of caps 015. Caps 015 are operable for removable attachment to tips 014. Mixing tip 016 is operable for removable attachment to tips 014. In FIG. 4, the nozzle of mixing tip 016 is configured to be at an angle, preferably a right angle.

FIG. 5 and FIG. 6 illustrate the same embodiments shown in FIG. 3 and FIG. 4, but showing caps 015 removed. In FIG. 6, the nozzle of mixing tip 016 is configured to be at an angle, preferably a right angle.

In one embodiment the present invention is used to touch up the roots of hair. The user removes multiple chamber syringe 010 from its packaging, removes both of cap 015 from each of tip 014, and secures mixing tip 016 to each of tip 014. The user then engages plunger 013 such that first composition 017 and second composition 018 are controllably released through tips 014 and become combined within mixing tip 016 and forms combined mixture 019. As combined mixture 019 is released through dispensing orifice 016a of mixing tip 016, the user positions dispensing orifice 016a of mixing tip 016 to the area of the roots of hair to be dyed, such that combined mixture 019 is applied to the roots of hair.

While the above description contains many specifics, these specifics should not be construed as limitations on the scope of the invention, but merely exemplifications of the disclosed embodiments. Those skilled in the art will envision other possible variations that are within the scope of the invention.

I claim:

1. A hair coloring kit for mixing together hair coloring materials comprising:
a multiple chambered syringe containing at least two ingredient filled chambers,
wherein said at least two ingredient filled chambers each has a plunger end and an egress end,
means for coloring hair, and
means for storing said hair coloring materials.

2. The hair coloring kit for mixing together hair coloring materials of claim 1, wherein said means for storing said hair coloring materials consists of caps removably attached to tips disposed at said egress end of each of said at least two ingredient filled chambers.

3. The hair coloring kit for mixing together hair coloring materials of claim 2, wherein said means for coloring hair consists of:
a plunger operable for simultaneously pushing ingredients out of said egress end of each of said at least two ingredient filled chambers, and
a mixing tip removably attached to each of said tips.

4. The hair coloring kit for mixing together hair coloring materials of claim 3, further comprising at least one pair of gloves and an instruction sheet.

5. The hair coloring kit for mixing together hair coloring materials of claim 4, further comprising at least one hair brush.

6. The hair coloring kit for mixing together hair coloring materials of claim 5, further comprising at least one mirror.

7. The hair coloring kit for mixing together hair coloring materials of claim 6, further comprising at least one container of hair dye remover.

8. The hair coloring kit for mixing together hair coloring materials of claim 7, further comprising a plurality of said multiple chambered syringe.

9. The hair coloring kit for mixing together hair coloring materials of claim 8, wherein said multiple chambered syringe has two chambers.

10. The hair coloring kit for mixing together hair coloring materials of claim 3, further comprising traditional hair coloring components of dying a full head of hair.

11. The hair coloring kit for mixing together hair coloring materials of claim 10, further comprising at least one pair of gloves.

12. The hair coloring kit for mixing together hair coloring materials of claim 11, further comprising at least one hair brush.

13. The hair coloring kit for mixing together hair coloring materials of claim 12, further comprising at least one container of hair dye remover.

14. The hair coloring kit for mixing together hair coloring materials of claim 13, further comprising a plurality of said multiple chambered syringe.

15. A hair coloring kit for mixing together hair coloring materials comprising:
a syringe having a first chamber and a second chamber with said first chamber containing a developer and said second chamber containing a colorant,
wherein said first chamber has a first tip operable for controlled release of said developer,
wherein said second chamber has a second tip operable for controlled release of said colorant,
wherein said syringe has a plunger that is operable for simultaneously pushing said developer through said first tip and said colorant through said second tip,
a mixing tip removably attached to said first tip and said second tip,
a first cap operable for removable attachment to said first tip, and
a second cap operable for removable attachment to said second tip.

16. The hair coloring kit for mixing together hair coloring materials of claim 14, further comprising a pair of gloves, a hair brush, a container of hair dye remover, instructions, and a mirror.

17. A hair coloring kit for mixing together hair coloring materials comprising:
a syringe having a first chamber and a second chamber with said first chamber containing a developer and said second chamber containing a colorant,
wherein said first chamber has a first tip operable for controlled release of said developer,
wherein said second chamber has a second tip operable for controlled release of said colorant,
wherein said syringe has a plunger that is operable for simultaneously pushing said developer through said first tip and said colorant through said second tip, and
a mixing tip permanently affixed to said first tip and said second tip.

18. The hair coloring kit for mixing together hair coloring materials of claim 17, wherein said mixing tip is configured to be at an angle.

19. the hair coloring kit for mixing together hair coloring materials of claim 18, wherein said angle is a right angle.
